(19) United States
(12) Patent Application Publication
(10) Pub. No.: US 2023/0219884 A1
(43) Pub. Date: Jul. 13, 2023

(54) PREPARATION METHOD OF ACRYLONITRILE DIMER

(71) Applicant: LG CHEM, LTD., Seoul (KR)

(72) Inventors: Si Jung Park, Daejeon (KR); Wonseok Kim, Daejeon (KR); Yujin An, Daejeon (KR); Hyunchul Jung, Daejeon (KR); Sae Hume Park, Daejeon (KR); Wan Kyu Oh, Daejeon (KR)

(21) Appl. No.: 18/008,648

(22) PCT Filed: Oct. 7, 2021
(86) PCT No.: PCT/KR2021/013749

§ 371 Date: Dec. 6, 2022

(30) Foreign Application Priority Data
Oct. 12, 2020 (KR) 10-2020-0131025
Oct. 6, 2021 (KR) 10-2021-0132515

(51) Int. Cl. C07C 253/30; C07C 253/34; B01J 27/16

(52) U.S. Cl. CPC C07C 253/30; C07C 253/34; B01J 27/16

(57) ABSTRACT

According to the present disclosure, there is provided a preparation method of an acrylonitrile dimer capable of obtaining an acrylonitrile dimer with high purity and enhancing process efficiency and economic feasibility by easily recovering the catalyst used in the reaction.

### CROSS-REFERENCE TO RELATED APPLICATION(S)

The present application is a National Phase entry pursuant to 35 U.S.C. § 371 of International Application No. PCT/KR2021/013749 filed on Oct. 7, 2021, and claims priority to and the benefit of Korean Patent Application No. 10-2020-0131025 filed on Oct. 12, 2020 and No. 10-2021-0132515 filed on Oct. 6, 2021 with the Korean Intellectual Property Office, the disclosures of which are incorporated herein by reference in their entirety.

### FIELD

The present disclosure relates to a preparation method of an acrylonitrile dimer.

### BACKGROUND

An acrylonitrile dimer collectively refers to 1,4-dicyano-1-butene (DCB), 1,4-dicyano-2-butene, methylene glutaronitrile (MGN), etc., which are straight-chain compounds having six carbon atoms prepared by dimerization of acrylonitrile. Out of the dimers of acrylonitrile, 1,4-dicyano-1-butene (DCB) and 1,4-dicyano-2-butene are collectively called 1,4-dicyanobutene (DCB). The 1,4-dicyanobutene (DCB) may be converted into adiponitrile through a hydrogenation reaction, which is advantageously used as an intermediate for preparing hexamethylenediamine, a main monomer of nylon 66.

The acrylonitrile dimer may be prepared by reacting acrylonitrile in a solvent capable of donating protons in the presence of a phosphorus-based catalyst. The main product of the dimerization reaction is 1,4-dicyanobutene (DCB), and a small amount of methylene glutaronitrile (MGN) may be prepared as a by-product. In a reaction mixture after the reaction, components such as MGN, unreacted acrylonitrile, catalyst, solvent, etc., are mixed together with DCB as the main product, and a pure acrylonitrile dimer may be obtained through a purification process.

Since the catalyst used for the preparation of the acrylonitrile dimer is an expensive compound and greatly affects preparation costs, an attempt has been made to recover and reuse the catalyst after the reaction. Accordingly, as a method of purifying the reaction mixture after the acrylonitrile dimerization reaction, a method of separating an acrylonitrile dimer from the catalyst by distillation or extracting the catalyst from the reaction mixture by using an extraction solvent has been proposed.

However, in the case of the distillation method, since a boiling point of the acrylonitrile dimer is as high as about 254° C., distillation is performed at a high temperature, and thus side reactions may occur. In addition, in the case of the extraction method, the affinity between the acrylonitrile dimer and the catalyst is so high that separation thereof is difficult and a separate extraction solvent is required, and a post-process for recovering the catalyst from the extract after extraction is further required, thereby making an overall preparation process more complicated and causing a problem of rising costs.

Accordingly, there is a need for a method capable of efficiently separating the acrylonitrile dimer and the catalyst during the purification process.

### SUMMARY

In the present disclosure, there is provided a preparation method of an acrylonitrile dimer capable of efficiently separating a phosphorus-based catalyst from an acrylonitrile dimer to enhance process efficiency and economic feasibility.

According to one embodiment of the present disclosure, there is provided a preparation method of an acrylonitrile dimer including

a reaction process of preparing an acrylonitrile dimer by reacting a reaction mixture comprising: a hydrocarbon-based solvent; an alcohol having a boiling point higher than a boiling point of the hydrocarbon-based solvent capable of phase separation from an acrylonitrile dimer, and having two to six carbon atoms; and acrylonitrile, in a presence of a phosphorus-based catalyst,

a first purification process of distilling the reaction mixture to remove unreacted acrylonitrile and the hydrocarbon-based solvent after completion of the reaction process, and

a second purification process of stirring the reaction mixture from which the unreacted acrylonitrile and the hydrocarbon-based solvent are removed, allowing the resulting reaction mixture to stand for phase separation, and separating an alcohol phase including the phosphorus-based catalyst and an acrylonitrile dimer phase. According to the present disclosure, an acrylonitrile dimer can be obtained with high purity, and a catalyst used for the reaction can be easily recovered, thereby enhancing the efficiency and economic feasibility of a preparation process.

### DETAILED DESCRIPTION

The terminology used herein is for the purpose of describing particular embodiments only and is not intended to be limiting of the invention. The singular forms are intended to include the plural forms as well, unless the context clearly indicates otherwise. It will be further understood that the terms “include”, “have”, or “possess” when used in this specification, specify the presence of stated features, steps, components, or combinations thereof, but do not preclude the presence or addition of one or more other features, steps, components, or combinations thereof.

As the present invention can be variously modified and have various forms, specific embodiments thereof are shown by way of examples and will be described in detail. However, it is not intended to limit the present invention to the particular form disclosed and it should be understood that the present invention includes all modifications, equivalents, and replacements within the idea and technical scope of the present invention.

Hereinafter, the present invention will be described in detail.

The preparation method of an acrylonitrile dimer according to one embodiment of the present disclosure includes: a reaction process of preparing an acrylonitrile dimer by reacting a reaction mixture comprising: a hydrocarbon-based solvent; an alcohol having a boiling point higher than a boiling point of the hydrocarbon-based solvent capable of phase separation from an acrylonitrile dimer, and having two to six carbon atoms; and acrylonitrile, in a presence of a phosphorus-based catalyst,

a first purification process of distilling the reaction mixture to remove unreacted acrylonitrile and the hydrocarbon-based solvent after completion of the reaction process, and

a second purification process of stirring the reaction mixture from which the unreacted acrylonitrile and the hydrocarbon-based solvent are removed, allowing the resulting reaction mixture to stand for phase separation, and separating an alcohol phase including the phosphorus-based catalyst and an acrylonitrile dimer phase.

In a conventional preparation method of an acrylonitrile dimer, a method of distilling the acrylonitrile dimer per se or extracting the catalyst from the reaction mixture by using a separate extraction solvent has been used to separate the acrylonitrile dimer and the catalyst after the dimerization reaction. However, the above method has problems such as complicated process, requiring a lot of manufacturing equipment, high costs, poor purification efficiency, etc.

Accordingly, in the preparation method of an acrylonitrile dimer of the present disclosure, an alcohol having a boiling point higher than that of the hydrocarbon-based solvent and capable of phase separation from the acrylonitrile dimer is used as a co-solvent together with the hydrocarbon-based solvent, thereby enhancing the efficiency and economic feasibility of the purification process.

In other words, the alcohol used in the present disclosure serves as a proton-donating solvent during the dimerization reaction of acrylonitrile, and has a boiling point higher than that of the hydrocarbon-based solvent. Therefore, it is not volatilized together in a first purification process of removing the unreacted acrylonitrile and the hydrocarbon-based solvent after the dimerization reaction. In addition, since the alcohol causes the phase separation from the acrylonitrile dimer, the alcohol may serve as an extraction solvent for extracting the catalyst in a second purification process. As such, the alcohol is both a reaction solvent and an extraction solvent at the same time, and thus the catalyst included in an alcohol phase after extraction may be recycled to a reaction step together with the alcohol without a separate purification process.

Thus, according to the present disclosure, side reactions caused by distillation at a high temperature may be prevented, the use of a separate extraction solvent and a removal process thereof are unnecessary, and the acrylonitrile dimer and the catalyst may be separated by a simplified process compared to the conventional method, thereby remarkably enhancing the efficiency, convenience and economic feasibility of the process.

As the alcohol, an aliphatic, alicyclic, or aromatic alcohol having two to six carbon atoms may be used. As one example, the alcohol used herein may be cyclohexanol, ethylene glycol, or a combination thereof, preferably cyclohexanol or ethylene glycol, and more preferably cyclohexanol.

In addition, the alcohol may have a boiling point higher than that of the hydrocarbon-based solvent by at least 40° C., and preferably at least 50° C. or at least 60° C.

When there is a little difference in boiling points between the alcohol and the hydrocarbon-based solvent, the alcohol may be distilled together in the first purification process without any remaining alcohol to be used as an extraction solvent in the second refining process, and thus it is preferable that the boiling points differ by 50° C. or more. The upper limit of the difference in boiling points between the alcohol and the hydrocarbon-based solvent may not be particularly limited, but may be, for example, 180° C. or less, 110° C. or less, or 90° C. or less.

In addition, the alcohol may preferably have a density of 0.965 g/cm³ or less, or 0.962 g/cm³ or less, and 1.05 g/cm³ or more, or 1.10 g/cm³ or more. Most of the dimers produced by the dimerization reaction of acrylonitrile may be 1,4-dicyanobutene having a density of about 1.0 g/cm³, and thus the phase separation from the acrylonitrile dimer may smoothly occur when the alcohol satisfies the above range of density.

Meanwhile, the alcohol may need to be capable of extracting the phosphorus-based catalyst from the acrylonitrile dimer, and thus the affinity for the phosphorus-based catalyst may need to be higher than that of the acrylonitrile dimer. Accordingly, an alcohol-adiponitrile partition coefficient (K_{ROH/ADN}) of the phosphorus-based catalyst represented by the following Equation 1 at 50° C. under a normal pressure (760 torr) may be 1.0 or more, and preferably 1.07 or more, 1.35 or more, or 1.5 or more. The upper limit of the partition coefficient may not be particularly limited, but may be, for example, 6.0 or less, 5.0 or less, or 4.8 or less.

K_{ROH/ADN}=C_{ROH}/C_{ADN} [Equation 1]

in above Equation 1,

C_{ROH} is a percent concentration of the phosphorus-based catalyst in alcohol, and

C_{ADN} is a percent concentration of the phosphorus-based catalyst in adiponitrile.

The hydrocarbon-based solvent may be used as a main solvent for the acrylonitrile dimerization reaction. As the hydrocarbon-based solvent, aromatic hydrocarbons, aliphatic hydrocarbons, alicyclic hydrocarbons, etc., having 5 to 15 carbon atoms may be used.

It is preferable that the hydrocarbon-based solvent may have a boiling point of 115° C. or less, or 110° C. or less and 20° C. or more, 30° C. or more, or 50° C. or more, so as to be smoothly removed by distillation together with unreacted acrylonitrile monomers (boiling point of 77° C.) after the completion of the acrylonitrile dimerization reaction. The hydrocarbon-based solvent may be at least one selected from the group consisting of benzene, toluene, n-pentane, n-hexane, cyclopentane and cyclohexane.

According to one preferred embodiment, toluene may be used as the hydrocarbon-based solvent, and cyclohexanol or ethylene glycol may be used as the alcohol. Toluene has a boiling point of about 110° C. and may be volatilized by distillation under relatively mild conditions. Cyclohexanol and ethylene glycol have a boiling point higher than that of toluene by 50° C. or more, and thus may not be volatilized in the first purification process. In addition, cyclohexanol and ethylene glycol may have a phase separation from the acrylonitrile dimer, and have a high solubility to the phosphorus-based catalyst. Thus, they may be suitably used as an extraction solvent for separating the acrylonitrile dimer and the catalyst in the second purification process.

When the amount of proton donating solvent is insufficient during the acrylonitrile dimerization reaction, the polymerization reaction of acrylonitrile may be accelerated. Accordingly, the alcohol may be used 3% by volume or more, 5% by volume or more, or 10% by volume or more, and 50% by volume or less, 40% by volume or less, or 30% by volume or less based on 100% by volume of the hydrocarbon-based solvent in the reaction process. When the content range is satisfied, the acrylonitrile dimerization reaction and the extraction process after the reaction may be smoothly performed.

The phosphorus-based catalyst used in the acrylonitrile dimerization reaction of the present disclosure may be an organophosphorus (III) compound containing at least one hydrocarbyl; and at least one alkoxy or cycloalkoxy.

As one example, the phosphorus-based catalyst may be a phosphinite-based compound represented by (R)₂P(OR′) or a phosphonite-based compound represented by (R)P(OR′)₂. In the above formulas (R)₂P(OR′) and (R)P(OR′)₂, R and R′ may each independently be C1-20 alkyl, C6-20 aryl, C7-20 alkylaryl, C7-20 aralkyl, or C3-20 alkyl. Preferably, R may each independently be C1-20 alkyl or C6-20 aryl, and R′ may be each independently be C1-20 alkyl.

Preferably, the phosphorus-based catalyst used herein may be at least one selected from the group consisting of ethyl diphenylphosphinite, isopropyl diphenylphosphinite, ethyl phenylethyl phosphinite, and isopropyl ditolyl phosphinite.

In the reaction process, the phosphorus-based catalyst may be included in an amount of 2 mol % or more, 3 mol % or more, or 4 mol % or more, and 8 mol % or less, 7 mol % or less, or 6 mol % or less based on 100 mol % of the acrylonitrile monomer. When the phosphorus-based catalyst is used within the above molar content with respect to the acrylonitrile monomer, side reactions may be suppressed while the acrylonitrile dimerization reaction smoothly proceeds.

In the preparation method of an acrylonitrile dimer of the present disclosure, the reaction process for dimerizing acrylonitrile may be performed at 0° C. to 70° C. under a normal pressure (700 to 800 torr), and preferably at 10° C. to 50° C., or 20° C. to 40° C. When a reaction temperature is less than 0° C., a reaction rate may be low and thus productivity may be reduced, and when the reaction temperature exceeds 70° C., the reaction rate may become excessively high, and thus the acrylonitrile monomer and dimer may be polymerized into a polymer.

When the reaction process is completed, a first purification process of distilling the reaction mixture to remove the unreacted acrylonitrile and the hydrocarbon-based solvent, having a relatively low boiling point, is performed. In this case, the distilled unreacted acrylonitrile and hydrocarbon-based solvent may be recovered and reused in the reactor.

The conditions of the first purification process may be adjusted according to the boiling point of the hydrocarbon-based solvent used. In one embodiment, the first purification process may be performed under a pressure of 760 torr or less, 400 torr or less, 100 torr or less, or 10 torr or less. The temperature of the first purification process may be appropriately adjusted according to pressure conditions, and may be, for example, in the range of 100° C. or less, 70° C. or less, or 60° C. or less, and 10° C. or more, or 30° C. or more. Under such conditions, only the unreacted acrylonitrile and hydrocarbon-based solvent may be selectively removed without evaporation of the alcohol.

Then, a second purification process of stirring the reaction mixture from which the unreacted acrylonitrile and the hydrocarbon-based solvent are removed, and then subjecting the resulting mixture to phase separation to separate an alcohol phase and an acrylonitrile dimer phase is performed.

Since the alcohol has a boiling point higher than that of the hydrocarbon-based solvent and thus most thereof is not volatilized in the first purification process, the phosphorus-based catalyst may be extracted by using alcohol present in the reaction mixture without further adding alcohol.

The second purification process may be performed at a temperature of 10 to 30° C., or 20 to 25° C. When the temperature of the second purification process is too high, the separation of the alcohol phase and the acrylonitrile dimer phase may not occur well, and the ratio of the phosphorus-based catalyst dissolved in the acrylonitrile dimer phase may increase to reduce the separation efficiency. Thus, it is preferable that the second purification process may be performed at room temperature.

Accordingly, after sufficiently mixing the reaction mixture within the above temperature range, the mixture is allowed to stand for phase separation, after which a supernatant and a lower layer solution are separated to complete the extraction.

In addition, in order to further enhance the recovery rate of the catalyst, the process of extraction by further adding the alcohol to the acrylonitrile dimer separated by the second purification process may be repeated.

Most of the acrylonitrile dimer phase separated from the alcohol phase through the above process may consist of 1,4-dicyanobutene (DCB) and may include a small amount of methylene glutaronitrile (MGN). Accordingly, in order to remove the MGN, the acrylonitrile dimer phase is further purified and a partial hydrogenation reaction is performed to prepare adiponitrile, a precursor of hexamethylenediamine.

Meanwhile, the alcohol phase containing the phosphorus-based catalyst may be recovered and reused in a reaction vessel. In the present disclosure, the alcohol used as a co-solvent in the reaction step is used in the second purification process for extracting the phosphorus-based catalyst without using a separate extraction solvent, and thus the process of removing the extraction solvent for reuse of the catalyst may be unnecessary. In addition, not only the catalyst but also the alcohol as the co-solvent may be reused together, thereby reducing preparation costs and increasing process efficiency.

Hereinafter, preferred examples will be suggested for better understanding of the present invention, but the following examples are provided only for the purpose of illustrating the present invention, and it is apparent to those having ordinary skill in the art that various changes and modifications are possible within the scope and technical ideas of the present invention and such changes and modifications are within the appended claims.

### EXAMPLES

### Example

<Evaluation of Partition Coefficient of Catalyst>

In order to confirm a partition coefficient of a phosphorus-based catalyst with respect to alcohol and acrylonitrile dimer, a following experiment was performed assuming that unreacted acrylonitrile and hydrocarbon-based solvent were removed from a reaction mixture through a first purification process.

(1) 0.634 g (25 wt %) of ethyl diphenylphosphinite (Ph₂POEt) and 2 ml (1.902 g, 75 wt %) of adiponitrile (ADN) were mixed and used as a simulated solution of the reaction mixture from which the unreacted acrylonitrile and the hydrocarbon-based solvent were removed after the first purification step.

(2) The 2 ml of the alcohol shown in Table 1 below was added to the simulated solution, and stirred while being heated to 50° C.

(3) After stirring at 50° C. for five minutes, stirring was stopped and allowed to stand.

(4) After phase separation, 0.25 ml of the supernatant and the lower layer solution was obtained, respectively, and components were analyzed by gas chromatography (GC, SIMADAZU, GC-2030). In this case, a detector was FID and set to 350° C., and HP-5MS was used for a column and set to 40-280° C. A temperature of an injection port was 260° C. and N2 was used as a mobile phase.

(5) The percent concentration of the catalyst included in each layer (ADN layer and alcohol layer) and the partition coefficient calculated therefrom are shown in table 1 below.

TABLE 1

| | Catalyst | Catalyst | |
|---|---|---|---|
| | concentration | concentration | Partition |
| | in alcohol layer | in ADN layer | coefficient |
| Alcohol | (C_{ROH}) | (C_{ADN}) | (C_{ROH}/C_{ADN}) |
| Cyclohexanol | 0.1628 | 0.1037 | 1.57 |
| Ethylene glycol | 0.1486 | 0.1077 | 1.38 |
| 1-octanol | 0.1303 | 0.1616 | 0.81 |
| 1-nonanol | 0.0778 | 0.1693 | 0.46 |

| | |
|---|---|
| Isopropanol | Layer separation from ADN does not occur well. |

<Preparation of Acrylonitrile Dimer>

### Example 1

An acrylonitrile dimerization reaction was performed as follows by using toluene as a solvent, cyclohexanol (boiling point of 161.8° C. and density of 0.962 g/cm³) as an alcohol co-solvent, and ethyl diphenylphosphinite (Ph₂POEt) as a catalyst.

Toluene, acrylonitrile, and cyclohexanol were added to a 1 L reactor at a volume ratio of 10:3:3, and ethyl diphenylphosphinite was added in an amount of 5 mol % based on the acrylonitrile. The mixture was reacted while being stirred at room temperature (25° C.) for 24 hours.

After completion of the reaction, the reaction mixture was distilled at a temperature of 60° C. and under a pressure of 10 torr to remove toluene and unreacted acrylonitrile from the reaction mixture. After that, the reaction mixture was stirred to mix, and then the stirring was stopped and allowed to stand for phase separation. After the phase separation was completed, the supernatant and the lower layer solution were separated, and the components of the supernatant and the lower layer solution were analyzed by using gas chromatography (GC, SIMADAZU, GC-2030). The detector was FID and set to 350° C., and HP-5MS was used for a column and set to 40-280° C. A temperature of an injection port was 260° C., N2 was used as a mobile phase, and 1 ml of the sample was injected.

As a result of the analysis, a detection amount was calculated compared to a sample input amount. It was confirmed that the lower layer solution includes 78.9 wt % of 1,4-dicyanobutene (DCB), 2.1 wt % of methylene glutaronitrile (MGN), 11.9 wt % of cyclohexanol, and 7.1 wt % of ethyl diphenylphosphinite, and it was also confirmed that the supernatant includes 81.3% of cyclohexanol, 10.3 wt % of ethyl diphenylphosphinite, 8.2 wt % of 1,4-dicyanobutene (DCB), and 0.2 wt % of methylene glutaronitrile (MGN).

### Example 2

An acrylonitrile dimerization reaction was performed as follows by using toluene as a solvent, ethylene glycol (boiling point of 197° C. and density of 1.110 g/cm³) as an alcohol co-solvent, and ethyl diphenylphosphinite (Ph₂POEt) as a catalyst.

Toluene, acrylonitrile, and ethylene glycol were added to a 1 L reactor at a volume ratio of 10:3:3, and ethyl diphenylphosphinite was added in an amount of 5 mol % based on the acrylonitrile. The mixture was reacted while being stirred at room temperature (25° C.) for 24 hours.

Thereafter, the first and second purification processes were performed in the same manner as in Example 1, and GC analysis was performed.

As a result of the analysis, a detection amount was calculated compared to a sample input amount. It was confirmed that the lower layer solution includes 76.9 wt % of ethylene glycol, 9.1 wt % of ethyl diphenylphosphinite, 13.6 wt % of 1,4-dicyanobutene (DCB), and 0.4 wt % of methylene glutaronitrile (MGN), and the supernatant includes 67.2 wt % of 1,4-dicyanobutene (DCB), 1.8 wt % of methylene glutaronitrile (MGN), 23.7 wt % of ethylene glycol, and 7.3 wt % of ethyl diphenylphosphinite.

### Comparative Example 1

The acrylonitrile dimerization reaction was performed as follows by using toluene as a solvent, isopropyl alcohol (boiling point of 82.5° C. and density of 0.786 g/cm³) and formamide (boiling point of 210° C. and density of 1.130 g/cm³) as a co-solvent, and ethyl diphenylphosphinite as a catalyst.

Toluene, acrylonitrile, isopropyl alcohol and formamide were added to a 1 L reactor at a volume ratio of 10:3:1:1, and ethyl diphenylphosphinite was added in an amount of 5 mol % based on the acrylonitrile. The mixture was reacted while being stirred at room temperature (25° C.) for 24 hours.

After completion of the reaction, the reaction mixture was distilled at a temperature of 60° C. and under a pressure of 10 torr to remove toluene, isopropyl alcohol and unreacted acrylonitrile from the reaction mixture. After that, the reaction mixture was stirred to mix, and then the stirring was stopped to stand, but phase separation of formamide and DCB did not occur.

### Comparative Example 2

An acrylonitrile dimerization reaction was performed as follows by using toluene as a solvent, octanol (boiling point of 185° C. and density of 0.824 g/cm³) as an alcohol co-solvent, and ethyl diphenylphosphinite (Ph₂POEt) as a catalyst.

Toluene, acrylonitrile, and octanol were added to a 1 L reactor at a volume ratio of 10:3:3, and ethyl diphenylphosphinite was added in an amount of 5 mol % based on the acrylonitrile. The mixture was reacted while being stirred at room temperature (25° C.) for 24 hours.

Thereafter, the first and second purification processes were performed in the same manner as in Example 1, and GC analysis was performed.

As a result of the analysis, a detection amount was calculated compared to a sample input amount. It was confirmed that the lower layer solution includes 11.1 wt % of octanol, 10.5 wt % of ethyl diphenylphosphinite, 75.8 wt % of 1,4-dicyanobutene (DCB), and 2.6 wt % of methylene glutaronitrile (MGN), and the supernatant includes 9.2 wt % of 1,4-dicyanobutene (DCB), 0.3 wt % of methylene glutaronitrile (MGN), 81.9 wt % of octanol, and 8.6 wt % of ethyl diphenylphosphinite.

### Comparative Example 3

An acrylonitrile dimerization reaction was performed as follows by using toluene as a solvent, nonanol (boiling point of 214° C. and density of 0.827 g/cm³) as an alcohol co-solvent, and ethyl diphenylphosphinite (Ph₂POEt) as a catalyst.

Toluene, acrylonitrile, and nonanol were added to a 1 L reactor at a volume ratio of 10:3:3, and ethyl diphenylphosphinite was added in an amount of 5 mol % based on the acrylonitrile. The mixture was reacted while being stirred at room temperature (25° C.) for 24 hours.

Thereafter, the first and second purification processes were performed in the same manner as in Example 1, and GC analysis was performed.

As a result of the analysis, a detection amount was calculated compared to a sample input amount. It was confirmed that the supernatant includes 85.4 wt % of nonanol, 5.8 wt % of ethyl diphenylphosphinite, 8.5 wt % of 1,4-dicyanobutene (DCB), and 0.3 wt % of methylene glutaronitrile (MGN), and the lower layer solution includes 74.6 wt % of 1,4-dicyanobutene (DCB), 2.2 wt % of methylene glutaronitrile (MGN), 10.3 wt % of nonanol, and 12.9 wt % of ethyl diphenylphosphinite.

1. A preparation method of an acrylonitrile dimer, the method comprising:
a reaction process of preparing an acrylonitrile dimer by reacting a reaction mixture comprising: a hydrocarbon-based solvent; an alcohol having a boiling point higher than a boiling point of the hydrocarbon-based solvent capable of phase separation from an acrylonitrile dimer, and having two to six carbon atoms; and acrylonitrile, in a presence of a phosphorus-based catalyst,
a first purification process of distilling the reaction mixture to remove unreacted acrylonitrile and the hydrocarbon-based solvent after completion of the reaction process, and
a second purification process of stirring the reaction mixture from which the unreacted acrylonitrile and the hydrocarbon-based solvent are removed, allowing the resulting reaction mixture to stand for phase separation, and separating an alcohol phase comprising the phosphorus-based catalyst and an acrylonitrile dimer phase.

2. The preparation method of an acrylonitrile dimer of claim 1,
wherein a boiling point of the alcohol is higher than a boiling point of the hydrocarbon-based solvent by 40° C. or more.

3. The preparation method of an acrylonitrile dimer of claim 1,
wherein the alcohol is cyclohexanol, ethylene glycol, or a combination thereof.

4. The preparation method of an acrylonitrile dimer of claim 1,
wherein the hydrocarbon-based solvent has a boiling point of 115° C. or less.

5. The preparation method of an acrylonitrile dimer of claim 1,
wherein the hydrocarbon-based solvent is at least one selected from the group consisting of benzene, toluene, n-pentane, n-hexane, cyclopentane and cyclohexane.

6. The preparation method of an acrylonitrile dimer of claim 1,
wherein the hydrocarbon-based solvent is toluene and the alcohol is cyclohexanol.

7. The preparation method of an acrylonitrile dimer of claim 1,
wherein the alcohol is used in an amount of 3% by volume to 50% by volume based on 100% by volume of the hydrocarbon-based solvent in the reaction process.

8. The preparation method of an acrylonitrile dimer of claim 1,
wherein the phosphorus-based catalyst is at least one selected from the group consisting of ethyl diphenylphosphinite, isopropyl diphenylphosphinite, ethyl phenylethyl phosphinite, and isopropyl ditolyl phosphinite.

9. The preparation method of an acrylonitrile dimer of claim 1,
wherein the unreacted acrylonitrile and the hydrocarbon-based solvent separated in the first purification process; and the alcohol phase comprising the phosphorus-based catalyst separated in the second purification process are recycled to the reaction process.
